(19) United States
(12) Patent Application Publication
(10) Pub. No.: US 2025/0207122 A1
(43) Pub. Date: Jun. 26, 2025

(54) GENERATION OF A LARGE LIBRARY OF BARCODED BEADS USING COMBINATORIAL SPLIT-POOL STRATEGY AND CHEMICAL LIGATION

(71) Applicant: ILLUMINA, INC., San Diego, CA (US)

(72) Inventors: Yin Nah Teo, Singapore (SG); Xiangyuan Yang, Singapore (SG); Wei Wei, Singapore (SG); Min Yen Lee, Singapore (SG)

(21) Appl. No.: 18/849,248

(22) PCT Filed: Dec. 28, 2023
(86) PCT No.: PCT/US23/86196

§ 371 Date: Sep. 20, 2024

Related U.S. Application Data
(60) Provisional application No. 63/477,747, filed on Dec. 29, 2022.

(51) Int. Cl. C12N 15/10

(52) U.S. Cl. CPC C12N 15/1065; C12N 15/1096

(57) ABSTRACT

The present disclosure is generally directed to methods of generating highly diverse barcoded bead pools via combinatorial split-pool strategies.

### CROSS-REFERENCE TO RELATED APPLICATIONS

This application claims the priority benefit under 35 U.S.C. § 119 (e) of U.S. Provisional Application No. 63/477,747, filed Dec. 29, 2022, which is incorporated herein by reference in its entirety.

### BACKGROUND

Spatially resolved genomics and transcriptomics are areas of high interest but are limited by the challenge of generating a diverse bead array comprising beads having unique oligonucleotide sequences relative to each other.

### SUMMARY

Bead arrays may be used with spatial genomics and transcriptomics, but one drawback is having an insufficient number of unique bead types (i.e., beads comprising unique nucleotide sequences). Typically, multiple beads in a bead array comprise the same nucleotide sequence which leads to informatics collisions. To enable use of a bead array as a substrate to capture spatial transcriptomics information of tissue, each bead on the array needs to have a unique barcode so that the attached oligonucleotide can be identified as a unique sequence in the bead array decoding process.

To be compatible with existing bead array substrate configuration, a large number (e.g., 50-500 million) of bead types are required. To achieve such a large number of bead types, the present disclosure provides combinatorial split-pool methods, wherein during each split round chemical ligation is performed to attach a new oligonucleotide sequence, thereby expanding the barcode library.

Accordingly, in some aspects the disclosure provides a method of generating a bead array comprising: (i) attaching a first oligonucleotide to a first population of beads, attaching a second oligonucleotide to a second population of beads, and attaching a third oligonucleotide to a third population of beads, wherein the first oligonucleotide, the second oligonucleotide, and the third oligonucleotide comprise unique nucleotide sequences relative to each other; (ii) combining the first population of beads, the second population of beads, and the third population of beads to create a first pool; (iii) splitting the first pool into a first plurality of aliquots; (iv) in a first aliquot of the first plurality of aliquots, chemically ligating a fourth oligonucleotide to: the first oligonucleotide in the presence of a first splint oligonucleotide, wherein the first splint oligonucleotide hybridizes to both the fourth oligonucleotide and to the first oligonucleotide; the second oligonucleotide in the presence of a second splint oligonucleotide, wherein the second splint oligonucleotide hybridizes to both the fourth oligonucleotide and to the second oligonucleotide; and the third oligonucleotide in the presence of a third splint oligonucleotide, wherein the third splint oligonucleotide hybridizes to both the fourth oligonucleotide and to the third oligonucleotide; wherein the first splint oligonucleotide, the second splint oligonucleotide, and the third splint oligonucleotide comprise unique nucleotide sequences relative to each other; in a second aliquot of the first plurality of aliquots, chemically ligating a fifth oligonucleotide to: the first oligonucleotide in the presence of a fourth splint oligonucleotide, wherein the fourth splint oligonucleotide hybridizes to both the fifth oligonucleotide and to the first oligonucleotide; the second oligonucleotide in the presence of a fifth splint oligonucleotide, wherein the fifth splint oligonucleotide hybridizes to both the fifth oligonucleotide and to the second oligonucleotide; and the third oligonucleotide in the presence of a sixth splint oligonucleotide, wherein the sixth splint oligonucleotide hybridizes to both the fifth oligonucleotide and to the third oligonucleotide; wherein the fourth splint oligonucleotide, the fifth splint oligonucleotide, and the sixth splint oligonucleotide comprise unique nucleotide sequences relative to each other; and in a third aliquot of the first plurality of aliquots, chemically ligating a sixth oligonucleotide to: the first oligonucleotide in the presence of a seventh splint oligonucleotide, wherein the seventh splint oligonucleotide hybridizes to both the sixth oligonucleotide and to the first oligonucleotide; the second oligonucleotide in the presence of an eighth splint oligonucleotide, wherein the eighth splint oligonucleotide hybridizes to both the sixth oligonucleotide and to the second oligonucleotide; and the third oligonucleotide in the presence of a ninth splint oligonucleotide, wherein the ninth splint oligonucleotide hybridizes to both the sixth oligonucleotide and to the third oligonucleotide, wherein the seventh splint oligonucleotide, the eighth splint oligonucleotide, and the ninth splint oligonucleotide comprise unique nucleotide sequences relative to each other; (v) combining the first aliquot of the first plurality of aliquots, the second aliquot of the first plurality of aliquots, and the third aliquot of the first plurality of aliquots to create a second pool; (vi) splitting the second pool into a second plurality of aliquots; (vii) in a first aliquot of the second plurality of aliquots, chemically ligating a seventh oligonucleotide to the fourth oligonucleotide in the presence of a tenth splint oligonucleotide, wherein the tenth splint oligonucleotide hybridizes to both the seventh oligonucleotide and to the fourth oligonucleotide; in a second aliquot of the second plurality of aliquots, chemically ligating an eighth oligonucleotide to the fifth oligonucleotide in the presence of an eleventh splint oligonucleotide, wherein the eleventh splint oligonucleotide hybridizes to both the eighth oligonucleotide and to the fifth oligonucleotide; and in a third aliquot of the second plurality of aliquots, chemically ligating a ninth oligonucleotide to the sixth oligonucleotide in the presence of a twelfth splint oligonucleotide, wherein the twelfth splint oligonucleotide hybridizes to both the ninth oligonucleotide and to the sixth oligonucleotide, and wherein the tenth splint oligonucleotide, the eleventh split oligonucleotide, and the twelfth splint oligonucleotide comprise unique nucleotide sequences relative to each other. In some embodiments, the method further comprises: (viii) combining the first aliquot of the second plurality of aliquots, the second aliquot of the second plurality of aliquots, and the third aliquot of the second plurality of aliquots to create a third pool; (ix) splitting the third pool into a third plurality of aliquots; (x) in a first aliquot of the third plurality of aliquots, chemically ligating a tenth oligonucleotide to the seventh oligonucleotide in the presence of a thirteenth splint oligonucleotide, wherein the thirteenth splint oligonucleotide hybridizes to both the tenth oligonucleotide and to the seventh oligonucleotide; in a second aliquot of the third plurality of aliquots, chemically ligating an eleventh oligonucleotide to the eighth oligonucleotide in the presence of a fourteenth splint oligonucleotide, wherein the fourteenth splint oligonucleotide hybridizes to both the eleventh oligonucleotide and to the eighth oligonucleotide; and in a third aliquot of the third plurality of aliquots, chemically ligating a twelfth oligonucleotide to the ninth oligonucleotide in the presence of a fifteenth splint oligonucleotide, wherein the fifteenth splint oligonucleotide hybridizes to both the twelfth oligonucleotide and to the ninth oligonucleotide, and wherein the thirteenth splint oligonucleotide, the fourteenth splint oligonucleotide, and the fifteenth splint oligonucleotide comprise unique nucleotide sequences relative to each other. In further aspects, the disclosure provides a method of generating a bead array comprising: (i) attaching a first oligonucleotide to one or more beads in a first population of beads, attaching a second oligonucleotide to one or more beads in a second population of beads, and attaching a third oligonucleotide to one or more beads in a third population of beads, wherein the first oligonucleotide, the second oligonucleotide, and the third oligonucleotide comprise unique nucleotide sequences relative to each other; (ii) combining the first population of beads, the second population of beads, and the third population of beads to create a first pool; (iii) splitting the first pool into a first plurality of aliquots; (iv) in a first aliquot of the first plurality of aliquots, chemically ligating a fourth oligonucleotide to the first oligonucleotide, the second oligonucleotide, and the third oligonucleotide; in a second aliquot of the first plurality of aliquots, chemically ligating a fifth oligonucleotide to the first oligonucleotide, the second oligonucleotide, and the third oligonucleotide; and in a third aliquot of the first plurality of aliquots, chemically ligating a sixth oligonucleotide to the first oligonucleotide, the second oligonucleotide, and the third oligonucleotide; (v) combining the first aliquot of the first plurality of aliquots, the second aliquot of the first plurality of aliquots, and the third aliquot of the first plurality of aliquots to create a second pool; (vi) splitting the second pool into a second plurality of aliquots; (vii) in a first aliquot of the second plurality of aliquots, chemically ligating a seventh oligonucleotide to the fourth oligonucleotide; in a second aliquot of the second plurality of aliquots, chemically ligating an eighth oligonucleotide to the fifth oligonucleotide; and in a third aliquot of the second plurality of aliquots, chemically ligating a ninth oligonucleotide to the sixth oligonucleotide. In some embodiments, the method further comprises (viii) combining the first aliquot of the second plurality of aliquots, the second aliquot of the second plurality of aliquots, and the third aliquot of the second plurality of aliquots to create a third pool; (ix) splitting the third pool into a third plurality of aliquots; (x) in a first aliquot of the third plurality of aliquots, chemically ligating a tenth oligonucleotide to the seventh oligonucleotide; in a second aliquot of the second plurality of aliquots, chemically ligating an eleventh oligonucleotide to the eighth oligonucleotide; and in a third aliquot of the second plurality of aliquots, chemically ligating a twelfth oligonucleotide to the ninth oligonucleotide. In some embodiments, the method results in the bead array comprising at least about 1×10⁶ unique oligonucleotide sequences attached thereto. In further embodiments, the method results in the bead array comprising at least about 1×10⁷ unique oligonucleotide sequences attached thereto. In still further embodiments, the method results in the bead array comprising at least about 1×10⁸ unique oligonucleotide sequences attached thereto. In some embodiments, the first, second, third, fourth, fifth, sixth, seventh, eighth, ninth, tenth, eleventh, and twelfth splint oligonucleotides have the same nucleotide sequence. In some embodiments, the first, second, third, fourth, fifth, sixth, seventh, eighth, ninth, tenth, eleventh, twelfth, thirteenth, fourteenth, and fifteenth splint oligonucleotides have the same nucleotide sequence. In various embodiments, an oligonucleotide comprising (i) a poly(T) or poly(A) nucleotide sequence; (ii) one or more gene-specific capture sequences; (iii) one or more universal capture sequences (e.g., a random nucleotide sequence or a semi-random nucleotide sequence), or a combination thereof, is chemically ligated to the end of each oligonucleotide on each bead in the bead array. In some embodiments, (i) comprises about 100 populations of beads, each population of beads comprising unique oligonucleotides attached thereto relative to every other population of beads. In further embodiments, (i) comprises about 13,600 populations of beads, and each population of beads comprises unique oligonucleotides attached thereto relative to every other population of beads. In some embodiments, the first oligonucleotide, the second oligonucleotide, and/or the third oligonucleotide are attached to the one or more beads through a cleavable linker. In various embodiments, prior to the chemically ligating a terminal deoxynucleotidyl transferase (TdT) enzyme is used to incorporate a 3′-modified nucleotide. In some embodiments, one or more of the first oligonucleotide, the second oligonucleotide, the third oligonucleotide, the fourth oligonucleotide, the fifth oligonucleotide, the sixth oligonucleotide, the seventh oligonucleotide, the eighth oligonucleotide, or the ninth oligonucleotide is a barcode. In further embodiments, each of the first oligonucleotide, the second oligonucleotide, the third oligonucleotide, the fourth oligonucleotide, the fifth oligonucleotide, the sixth oligonucleotide, the seventh oligonucleotide, the eighth oligonucleotide, and the ninth oligonucleotide is a barcode. In some embodiments, one or more of the tenth oligonucleotide, the eleventh oligonucleotide, or the twelfth oligonucleotide is a barcode. In further embodiments, each of the tenth oligonucleotide, the eleventh oligonucleotide, and the twelfth oligonucleotide is a barcode. In still further embodiments, the barcode is a spatial barcode.

### BRIEF DESCRIPTION OF THE DRAWINGS

FIG. 1 depicts a general schematic of an exemplary combinatorial split and pool method of the disclosure for bead pool generation. An oligonucleotide is ligated (e.g., chemically ligated) onto the end of the growing barcode chain at each split-pool step.

FIG. 2 shows various non-limiting examples of unnatural backbone linkages (1 to 7: triazole, 8: squaramide, 9: urea) that may be used for library synthesis.

FIG. 3 shows a) Phosphate or phosphate derivative linkages for chemical ligation b) Non-limiting examples of the type of chemistries that may be used to generate the phosphate derived backbone in chemical ligation.

FIG. 4 shows structures of various 3′ and 5′ modifications that may be used for unnatural but biocompatible backbones shown in FIG. 2.

FIG. 5 shows an illustrative workflow of barcode generation using TdT enzyme to incorporate a modified nucleotide that enables chemical ligation. SM=surface modification. CL=cleavable linker. “N” in the phrase “N no. of sequences for a unique barcode on bead” refers to the number of times the chemical ligation takes place.

FIG. 6 shows an illustrative workflow of barcode generation using only chemical ligation, without TdT. SM=surface modification. CL=cleavable linker. “N” in the phrase “N no. of sequences for a unique barcode on bead” refers to the number of times the chemical ligation takes place.

FIG. 7 depicts an illustrative workflow comprising protection of one reactive group (two chemical reactive handles with protection).

FIG. 8 depicts an illustrative workflow comprising two pairs of orthogonal reactive groups (four chemical reactive handles).

FIG. 9 depicts a schematic of ligation with a splint oligonucleotide. R1 and R2 are functional groups for chemical ligation.

FIG. 10 depicts non-limiting examples of chemical cleavable attachments that may be used for barcode attachment to beads and optional subsequent cleavage from beads for sequencing.

FIG. 11 depicts types of splint oligonucleotides contemplated by the disclosure. a) Universal bridge sequence. Nucleotide sequences A′ and B′ are universal sequences that hybridize to nucleotide sequences A and B of the universal bridge sequence. Nucleotide sequence C is unique Oligo Seq 1 sequence. Nucleotide sequence D is unique Oligo Seq 2 sequence. Nucleotide sequence E is unique Oligo Seq 3 sequence. b) Unique bridge sequence. Nucleotide sequences A′ and B′ are unique sequences that hybridize to nucleotide sequences A and B of a first unique bridge sequence. Similarly, nucleotide sequences G′ and H′ are unique nucleotide sequences that hybridize to nucleotide sequences G and H of a second unique bridge sequence. Nucleotide sequence A′, nucleotide sequence B′, nucleotide sequence G′, nucleotide sequence H′, nucleotide sequence F′, and nucleotide sequence l′ are each unique nucleotide sequences relative to each other. Nucleotide sequence C is unique Oligo Seq 1 sequence. Nucleotide sequence D is unique Oligo Seq 2 sequence. Nucleotide sequence E is unique Oligo Seq 3 sequence. R1 and R2 are functional groups for chemical ligation.

### DETAILED DESCRIPTION

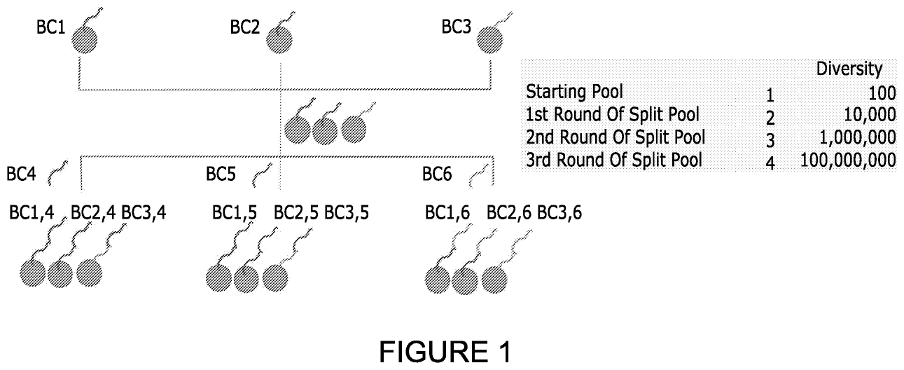

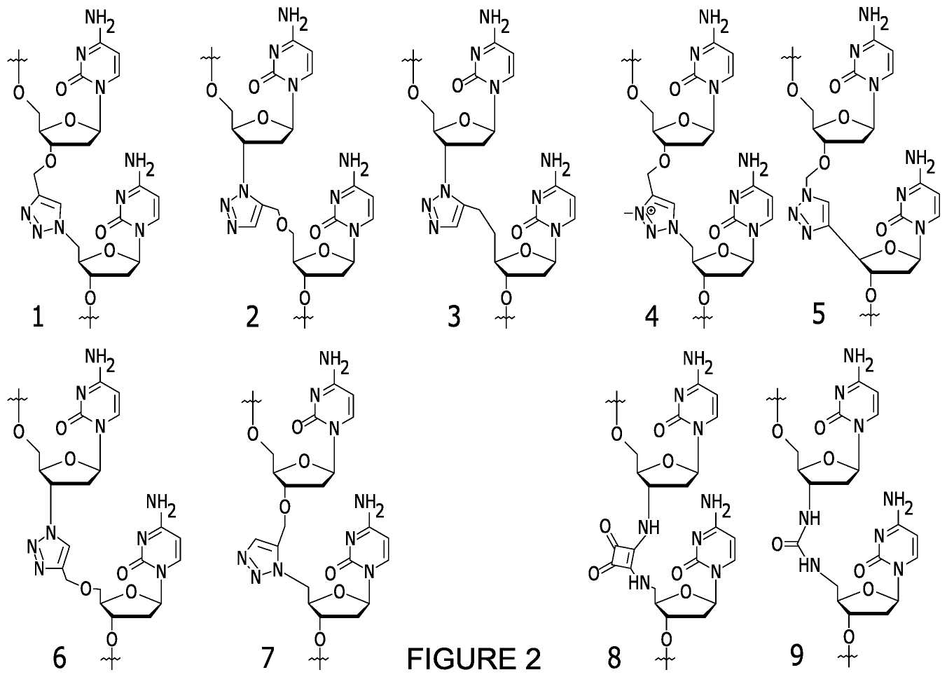

The present disclosure is generally directed to methods of generating a bead pool via combinatorial split-pool strategies. Oligonucleotides on bead arrays are typically synthesized in full prior to attachment to the beads. Generating a large pool of unique oligonucleotide barcoded beads, however, would require synthesizing an enormous variety of longer oligonucleotide sequences. Synthesis of large oligonucleotides poses significant challenges and often results in a reduced yield. Accordingly, the present disclosure provides methods of using combinatorial split pool and ligation (e.g., chemical ligation) to generate a large pool of unique oligonucleotide sequences. A general non-limiting schematic of a method of the disclosure is provided in FIG. 1.

### TERMS

As used in this specification and the enumerated paragraphs herein, the singular forms “a,” “an,” and “the” include plural reference unless the context clearly dictates otherwise.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

The term “bead” refers to a small body made of a rigid or semi-rigid material. The body can have a shape characterized, for example, as a sphere, oval, microsphere, or other recognized particle shape whether having regular or irregular dimensions. Example materials that are useful for beads include, without limitation, glass; plastic such as acrylic, polystyrene or a copolymer of styrene and another material, polypropylene, polyethylene, polybutylene, polyurethane or polytetrafluoroethylene (TEFLON®, from Chemours); polysaccharides or cross-linked polysaccharides such as agarose or Sepharose; nylon; nitrocellulose; resin; silica or silica-based materials including silicon and modified silicon; carbon-fiber, metal; inorganic glass; optical fiber bundle, or a variety of other polymers. Example beads include, without limitation, controlled pore glass beads, paramagnetic beads, thoria sol, Sepharose beads, nanocrystals and others known in the art as described, for example, in Microsphere Detection Guide from Bangs Laboratories, Fishers Ind. Beads may also be coated with a polymer that has a functional group that can attach to an oligonucleotide.

As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fibre bundles, and polymers. A particularly useful material is glass. Other suitable substrate materials may include polymeric materials, plastics, silicon, quartz (fused silica), boro float glass, silica, silica-based materials, carbon, metals including gold, an optical fibre or optical fibre bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength, such as one or more of the techniques set forth herein. Conversely, it may be desirable to select a material that does not pass radiation of a certain wavelength (e.g., being opaque, absorptive or reflective). This can be useful for formation of a mask to be used during manufacture of the structured substrate; or to be used for a chemical reaction or analytical detection carried out using the structured substrate. Other properties of a material that can be exploited are inertness or reactivity to certain reagents used in a downstream process; or ease of manipulation or low cost during a manufacturing process manufacture. Further examples of materials that can be used in the structured substrates or methods of the present disclosure are described in US Pat. App. Pub. No. 2012/0316086 A1 and 2013/0116153, the entire contents of each are incorporated by reference herein. In some embodiments, the solid support is a flow cell as described herein below.

In any of the aspects or embodiments of the disclosure, a solid support can include a collection of beads or other particles. The particles can be suspended in a solution or they can be located on the surface of a substrate. Examples of arrays having beads located on a surface include those wherein beads are located in wells such as a BeadChip array (Illumina Inc., San Diego Calif.), substrates used in sequencing platforms from 454 LifeSciences (a subsidiary of Roche, Basel Switzerland) or substrates used in sequencing platforms from Ion Torrent (a subsidiary of Life Technologies, Carlsbad Calif.). Other solid supports having beads located on a surface are described in U.S. Pat. Nos. 6,266,459; 6,355,431; 6,770,441; 6,859,570; 6,210,891; 6,258,568; or 6,274,320; US Pat. App. Publ. Nos. 2009/0026082 A1; 2009/0127589 A1; 2010/0137143 A1; or 2010/0282617 A1 or PCT Publication No. WO 00/63437, each of which is incorporated herein by reference. Several of the above references describe methods for attaching nucleic acids to beads prior to loading the beads in or on a solid support. It will, however, be understood that the ligated oligonucleotides can be made first and then attached to the beads which can then be loaded onto an array and used in a method set forth herein. In some embodiments, the ligated oligonucleotides are released from the beads and attached to a solid support (for example and without limitation, a flow cell). Accordingly, the present disclosure also contemplates the use of a flow cell. As used herein, the term “flow cell” is intended to mean a vessel having a chamber where a reaction can be carried out, an inlet for delivering reagents to the chamber and an outlet for removing reagents from the chamber. In some embodiments the chamber is configured for detection of the reaction that occurs in the chamber. For example, the chamber can include one or more transparent surfaces allowing optical detection of biological specimens, optically labeled molecules, or the like in the chamber. Exemplary flow cells include, but are not limited to those used in a nucleic acid sequencing apparatus such as flow cells for the Genome Analyzer®, MiSeq®, NextSeq® or HiSeq® platforms commercialized by Illumina, Inc. (San Diego, Calif.); or for the SOLID™ or Ion Torrent™ sequencing platform commercialized by Life Technologies (Carlsbad, Calif.). Exemplary flow cells and methods for their manufacture and use are also described, for example, in WO 2014/142841 A1; U.S. Pat. App. Pub. No. 2010/0111768 A1 and U.S. Pat. No. 8,951,781, each of which is incorporated herein by reference.

In some embodiments, the solid supports typically used for bead arrays are used without beads. For example, nucleic acids, such as the ligated oligonucleotides described herein, can be attached directly to the wells or to gel material in wells. Thus, the above references are illustrative of materials, compositions or apparatus that can be modified for use in the methods and compositions set forth herein.

A solid support used in a method set forth herein can include an array of beads, wherein different ligated oligonucleotides are attached to different beads in the array. In various embodiments, each bead can be attached to a different ligated oligonucleotide and the beads can be randomly distributed on the solid support in order to effectively attach the different nucleic acid probes to the solid support.

Optionally, the solid support can include wells having dimensions that accommodate no more than a single bead. In such a configuration, the beads may be attached to the wells due to forces resulting from the fit of the beads in the wells. It is also possible to use attachment chemistries or adhesives to hold the beads in the wells.

As described herein, ligated oligonucleotides that are attached to beads can comprise or consist of barcode sequences. According to methods provided herein, a population of beads can be configured such that each bead is attached to only one type of ligated oligonucleotide comprising a plurality of barcodes, and many different beads (each with a different ligated oligonucleotide) are present in the population.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules. The term can be similarly applied to proteins which are distinguishable as different from each other based on amino acid sequence differences.

By “complementary” is meant that an oligonucleotide comprises a sequence of nucleotides that can form a double-stranded structure by matching base-pairs with another oligonucleotide or part thereof. By “substantially complementary” is meant that the oligonucleotide has at least 85%, 90%, 95%, 98%, 99% or 100% overall sequence identity to the complementary sequence.

A bead array comprising oligonucleotide barcodes can also be used in a sequencing procedure, such as a sequencing-by-synthesis (SBS) technique. Briefly, SBS can be initiated by contacting the barcodes with one or more labeled nucleotides, DNA polymerase, etc. Those features where a primer is extended using the sequences comprising the barcode as a template will incorporate a labeled nucleotide that can be detected. Optionally, the labeled nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with an array produced by the methods of the present disclosure are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 or 7,405,281, and US Pat. App. Pub. No. 2008/0108082 A1, each of which is incorporated herein by reference.

As used herein, a “primer” is a nucleic acid molecule that can hybridize to a target sequence, such as an adapter attached to a library fragment. As one example, an amplification primer can serve as a starting point for template amplification and cluster generation. As another example, a synthesized nucleic acid (template) strand may include a site to which a primer (e.g., a sequencing primer) can hybridize in order to prime synthesis of a new strand that is complementary to the synthesized nucleic acid strand. Any primer can include any combination of nucleotides or analogs thereof. In some examples, the primer is a single-stranded oligonucleotide or polynucleotide. The primer length can be any number of bases long and can include a variety of non-natural nucleotides. In various embodiments, the sequencing primer is a short strand, ranging from 5 to 60 bases, from 10 to 60 bases, from 10 to 20 bases, from 10 to 30 bases, from 10 to 40 bases, from 10 to 50 bases, or from 20 to 40 bases.

As used herein, the term “unique molecular identifier” or “UMI” refers to a molecular tag, either random, non-random, or semi-random, that may be attached to a nucleic acid. When incorporated into a nucleic acid, a UMI can be used to correct for subsequent amplification bias by directly counting unique molecular identifiers (UMIs) that are sequenced after amplification. A UMI can be attached to similar nucleic acids, e.g., adapters, making each nucleic acid unique.

As used herein, the term “adapter” refers generally to any linear nucleic acid molecule that can be ligated to an oligonucleotide of the disclosure. In some embodiments, adapters include two reverse complementary oligonucleotides forming a double-stranded structure. In some embodiments, an adapter includes two oligonucleotides that are complementary at one portion and mismatched at another portion, forming a Y-shape or fork-shaped adapter that is double stranded at the complementary portion and has two floppy overhangs at the mismatched portion.

As used herein, the term “barcode” is intended to mean a series of nucleotides in an oligonucleotide that can be used to identify the oligonucleotide, a spatial address on a surface, a characteristic of the oligonucleotide, or a manipulation that has been carried out on the oligonucleotide. The barcode can be a naturally occurring nucleotide sequence or a nucleotide sequence that does not occur naturally in the organism from which the barcoded nucleic acid was obtained. A barcode sequence can be unique to a single nucleic acid species in a population or a barcode sequence can be shared by several different nucleic acid species in a population. For example, each nucleic acid capture probe in a population on a substrate for spatial capture of nucleic acids in a biological sample, e.g., a permeabilized tissue sample, a cell suspension, can include different barcode sequences from all other nucleic acid capture probes in the population. Alternatively, each nucleic acid probe in a population can include different barcode sequences from some or most other nucleic acid capture probes in a population. For example, each capture probe in a population can have a barcode that is present for several different capture probes in the population even though the capture probes with the common barcode differ from each other at other sequence regions along their length. In various embodiments, one or more barcode sequences that are used with a biological tissue are not present in the genome, transcriptome or other nucleic acids of the biological specimen. For example, barcode sequences can have less than 80%, 70%, 60%, 50% or 40% sequence identity to the nucleic acid sequences in a particular biological tissue.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single nucleic acid molecule having a particular sequence or a site can include several nucleic acid molecules having the same sequence (and/or complementary sequence, thereof). The sites of an array can be different features located on the same substrate. Exemplary features include without limitation, beads (or other particles) in or on a substrate, droplets, wells in a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “tissue” is intended to mean an aggregation of cells, and, optionally, intercellular matter. Typically the cells in a tissue are not free floating in solution and instead are attached to each other to form a multicellular structure. Exemplary tissue types include muscle, nerve, epidermal and connective tissues.

The terms “P5” and “P7” may be used when referring to examples of adapters. The terms “P5” (P5 prime) and “P7” (P7 prime) refer to the complement of P5 and P7, respectively. It will be understood that any suitable adapter can be used in the methods presented herein, and that the use of P5 and P7 are exemplary embodiments only. Uses of adapters such as P5 and P7 or their complements on flowcells are known in the art, as exemplified by the disclosures of WO 2007/010251, WO 2006/064199, WO 2005/065814, WO 2015/106941, WO 1998/044151, and WO 2000/018957, each of which is incorporated herein by reference in its entirety. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein.

As used herein, the terms “ligating”, “ligation” and their derivatives refer generally to the process for covalently linking two or more molecules together, for example covalently linking two or more nucleic acid molecules to each other. In some embodiments, ligation includes joining nicks between adjacent nucleotides of nucleic acids. In some embodiments, ligation includes forming a covalent bond between an end of a first oligonucleotide and an end of a second oligonucleotide. In some embodiments, the ligation can include forming a covalent bond between a 5′ phosphate group of one oligonucleotide and a 3′ hydroxyl group of a second oligonucleotide thereby forming a ligated nucleic acid molecule. A “ligated oligonucleotide” as used herein refers to an oligonucleotide that is produced following two or more rounds of a combinatorial split-pool method of the disclosure. Chemical ligation of oligonucleotides is further described herein.

As used herein, “hybridize” is intended to mean noncovalently associating a first oligonucleotide to a second oligonucleotide along the lengths of those polymers to form a double-stranded “duplex.” For instance, two DNA oligonucleotide strands may associate through complementary base pairing. The strength of the association between the first and second oligonucleotides increases with the complementarity between the sequences of nucleotides within those oligonucleotides. The strength of hybridization between oligonucleotides may be characterized by a temperature of melting (Tm) at which 50% of the duplexes have oligonucleotide strands that disassociate from one another. Oligonucleotides that are “partially” hybridized to one another means that they have sequences that are complementary to one another, but such sequences are hybridized with one another along only a portion of their lengths to form a partial duplex. Oligonucleotides with an “inability” to hybridize include those that are physically separated from one another such that an insufficient number of their bases may contact one another in a manner so as to hybridize with one another.

As used herein, the term “plurality” is intended to mean a population of two or more members, which may all be the same or two or more members may be different. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two. An upper limit of a plurality may be set, for example, by the theoretical diversity of bead types in an array.

As used herein, the term “attached” refers to the state of two things being joined, fastened, adhered, connected or bound to each other. For example, an oligonucleotide can be attached to a material, such as a bead, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, a “semi-random” nucleotide sequence comprises or consists of a partially pre-determined nucleotide sequence combined with a random nucleotide sequence.

### Oligonucleotides

An oligonucleotide is a polymer comprised of nucleotides. Oligonucleotides of the disclosure may be of any length and include, in various embodiments, DNA oligonucleotides, RNA oligonucleotides, analogs thereof, or a combination thereof. In any aspects or embodiments described herein, an oligonucleotide is single-stranded, double-stranded, or partially double-stranded.

Nucleotides may include naturally occurring nucleotides and functional analogs thereof. Examples of functional analogs are those that are capable of hybridizing to a nucleic acid in a sequence specific fashion or capable of being used as a template for replication of a particular nucleotide sequence. Naturally occurring nucleotides generally have a backbone containing phosphodiester bonds. An analog structure can have an alternate backbone linkage including any of a variety known in the art. Naturally occurring nucleotides generally have a deoxyribose sugar (e.g., found in DNA) or a ribose sugar (e.g., found in RNA). An analog structure can have an alternate sugar moiety including any of a variety known in the art. Nucleotides can include native or non-native bases. A native DNA can include one or more of adenine, thymine, cytosine and/or guanine, and a native RNA can include one or more of adenine, uracil, cytosine and/or guanine. Any non-native base may be used, such as a locked nucleic acid (LNA) and a bridged nucleic acid (BNA). Example modified nucleotides include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

Oligonucleotides contemplated by the disclosure also include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. Examples of universal bases include but are not limited to 5′-nitroindole-2′-deoxyriboside, 3-nitropyrrole, inosine and hypoxanthine.

### Methods

The present disclosure is generally directed to methods of generating a large library of barcoded beads using a combinatorial split-pool strategy and ligation (e.g., chemical ligation). In general, the methods comprise successive rounds of oligonucleotide (e.g., barcode) addition to a growing oligonucleotide chain. The successive rounds of oligonucleotide addition to a growing oligonucleotide chain may be performed in solution, or the successive rounds of oligonucleotide addition to a growing oligonucleotide chain may be performed directly on, e.g., a bead. Thus, during each “split” round, ligation (e.g., chemical ligation) is performed to attach a new oligonucleotide sequence (e.g., a barcode sequence), thereby expanding the library (e.g., barcode library). Accordingly, methods of the disclosure include at least one “split and pool” step of collecting pooled oligonucleotides (e.g., barcodes), distributing them into aliquots, and adding a further oligonucleotide (e.g., barcode), where the number of “split and pool” steps dictate the number of different oligonucleotides (e.g., barcodes) that are added to the growing oligonucleotide. This process results in unique oligonucleotide (e.g., barcode) combinations being produced. In various embodiments, one, two, three, four, five, six, seven, eight, nine, ten, or more split and pool rounds are performed. In various embodiments, a method of the disclosure results in the bead array comprising about, at least about, or less than about 1×10², 1×10³, 1×10⁴, 1×10⁵, 1×10⁶, 1×10⁷, 1×10⁸ or more unique oligonucleotide sequences attached thereto.

Accordingly, in some aspects the present disclosure provides a method of generating a bead array comprising (i) attaching a first oligonucleotide to a first population of beads, attaching a second oligonucleotide to a second population of beads, and attaching a third oligonucleotide to a third population of beads, wherein the first oligonucleotide, the second oligonucleotide, and the third oligonucleotide comprise unique nucleotide sequences relative to each other; (ii) combining the first population of beads, the second population of beads, and the third population of beads to create a first pool; (iii) splitting the first pool into a first plurality of aliquots; (iv) in a first aliquot of the first plurality of aliquots, chemically ligating a fourth oligonucleotide to: the first oligonucleotide in the presence of a first splint oligonucleotide, wherein the first splint oligonucleotide hybridizes to both the fourth oligonucleotide and to the first oligonucleotide; the second oligonucleotide in the presence of a second splint oligonucleotide, wherein the second splint oligonucleotide hybridizes to both the fourth oligonucleotide and to the second oligonucleotide; and the third oligonucleotide in the presence of a third splint oligonucleotide, wherein the third splint oligonucleotide hybridizes to both the fourth oligonucleotide and to the third oligonucleotide; wherein the first splint oligonucleotide, the second splint oligonucleotide, and the third splint oligonucleotide comprise unique nucleotide sequences relative to each other; in a second aliquot of the first plurality of aliquots, chemically ligating a fifth oligonucleotide to: the first oligonucleotide in the presence of a fourth splint oligonucleotide, wherein the fourth splint oligonucleotide hybridizes to both the fifth oligonucleotide and to the first oligonucleotide; the second oligonucleotide in the presence of a fifth splint oligonucleotide, wherein the fifth splint oligonucleotide hybridizes to both the fifth oligonucleotide and to the second oligonucleotide; and the third oligonucleotide in the presence of a sixth splint oligonucleotide, wherein the sixth splint oligonucleotide hybridizes to both the fifth oligonucleotide and to the third oligonucleotide; wherein the fourth splint oligonucleotide, the fifth splint oligonucleotide, and the sixth splint oligonucleotide comprise unique nucleotide sequences relative to each other; and in a third aliquot of the first plurality of aliquots, chemically ligating a sixth oligonucleotide to: the first oligonucleotide in the presence of a seventh splint oligonucleotide, wherein the seventh splint oligonucleotide hybridizes to both the sixth oligonucleotide and to the first oligonucleotide; the second oligonucleotide in the presence of an eighth splint oligonucleotide, wherein the eighth splint oligonucleotide hybridizes to both the sixth oligonucleotide and to the second oligonucleotide; and the third oligonucleotide in the presence of a ninth splint oligonucleotide, wherein the ninth splint oligonucleotide hybridizes to both the sixth oligonucleotide and to the third oligonucleotide, wherein the seventh splint oligonucleotide, the eighth splint oligonucleotide, and the ninth splint oligonucleotide comprise unique nucleotide sequences relative to each other; (v) combining the first aliquot of the first plurality of aliquots, the second aliquot of the first plurality of aliquots, and the third aliquot of the first plurality of aliquots to create a second pool; (vi) splitting the second pool into a second plurality of aliquots; (vii) in a first aliquot of the second plurality of aliquots, chemically ligating a seventh oligonucleotide to the fourth oligonucleotide in the presence of a tenth splint oligonucleotide, wherein the tenth splint oligonucleotide hybridizes to both the seventh oligonucleotide and to the fourth oligonucleotide; in a second aliquot of the second plurality of aliquots, chemically ligating an eighth oligonucleotide to the fifth oligonucleotide in the presence of an eleventh splint oligonucleotide, wherein the eleventh splint oligonucleotide hybridizes to both the eighth oligonucleotide and to the fifth oligonucleotide; and in a third aliquot of the second plurality of aliquots, chemically ligating a ninth oligonucleotide to the sixth oligonucleotide in the presence of a twelfth splint oligonucleotide, wherein the twelfth splint oligonucleotide hybridizes to both the ninth oligonucleotide and to the sixth oligonucleotide. In various embodiments, step (i) comprises about, at least about, or less than about 10, 20, 50, 100, 200, 500, 1,000, 2,000, 5,000, 10,000, 13,600, 15,000, 20,000, 50,000, 100,000, 200,000, 500,000, 700,000, 1×10⁶, 2×10⁶, 3×10⁶, 4×10⁶, 5×10⁶, 6×10⁶, 7×10⁶, 8×10⁶ or more populations of beads, and each population of beads comprises unique oligonucleotides attached thereto relative to every other population of beads.

In further aspects, the disclosure provides a method of generating a bead array comprising: (i) attaching a first oligonucleotide to one or more beads in a first population of beads, attaching a second oligonucleotide to one or more beads in a second population of beads, and attaching a third oligonucleotide to one or more beads in a third population of beads, wherein the first oligonucleotide, the second oligonucleotide, and the third oligonucleotide comprise unique nucleotide sequences relative to each other; (ii) combining the first population of beads, the second population of beads, and the third population of beads to create a first pool; (iii) splitting the first pool into a first plurality of aliquots; (iv) in a first aliquot of the first plurality of aliquots, chemically ligating a fourth oligonucleotide to the first oligonucleotide, the second oligonucleotide, and the third oligonucleotide; in a second aliquot of the first plurality of aliquots, chemically ligating a fifth oligonucleotide to the first oligonucleotide, the second oligonucleotide, and the third oligonucleotide; and in a third aliquot of the first plurality of aliquots, chemically ligating a sixth oligonucleotide to the first oligonucleotide, the second oligonucleotide, and the third oligonucleotide; (v) combining the first aliquot of the first plurality of aliquots, the second aliquot of the first plurality of aliquots, and the third aliquot of the first plurality of aliquots to create a second pool; (vi) splitting the second pool into a second plurality of aliquots; (vii) in a first aliquot of the second plurality of aliquots, chemically ligating a seventh oligonucleotide to the fourth oligonucleotide; in a second aliquot of the second plurality of aliquots, chemically ligating an eighth oligonucleotide to the fifth oligonucleotide; and in a third aliquot of the second plurality of aliquots, chemically ligating a ninth oligonucleotide to the sixth oligonucleotide. In various embodiments, step (i) comprises about, at least about, or less than about 10, 20, 50, 100, 200, 500, 1,000, 2,000, 5,000, 10,000, 13,600, 15,000, 20,000, 50,000, 100,000, 200,000, 500,000, 700,000, 1×10⁶, 2×10⁶, 3×10⁶, 4×10⁶, 5×10⁶, 6×10⁶, 7×10⁶, 8×10⁶ or more populations of beads, and each population of beads comprises unique oligonucleotides attached thereto relative to every other population of beads.

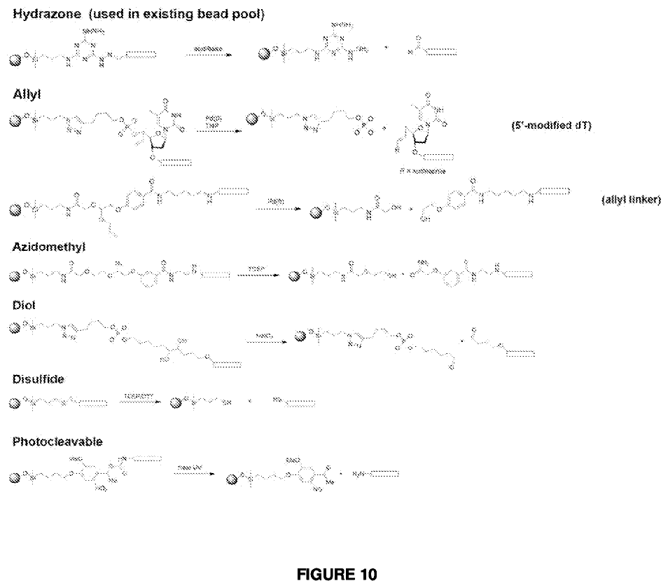

In any of the aspects or embodiments of the disclosure, the last oligonucleotide sequence (e.g., barcode sequence) that is added to the growing oligonucleotide chain comprises a unique molecular identifier (UMI). In any of the aspects or embodiments of the disclosure, the last oligonucleotide sequence (e.g., barcode sequence) that is added to the growing oligonucleotide chain comprises a poly(T) or poly(A) nucleotide sequence. In some embodiments, the last oligonucleotide sequence (e.g., barcode sequence) that is added to the growing oligonucleotide chain (which is, for example, a reverse transcription primer) is cleaved off from the bead surface for sequencing. Accordingly, in some embodiments the disclosure contemplates that a cleavable linkage is utilized between the beads and the barcoded primers. Cleavable linkers for use in such methods include, but are not limited to, the cleavable linkers described in Bioorg. Med. Chem. 2012, 20, 571-582 as well as the cleavable linkers depicted in FIG. 10.

Oligonucleotides for use in the methods described herein generally range from about 5 to about 100 nucleotides in length. In various embodiments, an oligonucleotide of the disclosure ranges from about 5 to about 100 nucleotides, from about 5 to about 90 nucleotides, from about 5 to about 80 nucleotides, from about 5 to about 70 nucleotides, from about 5 to about 60 nucleotides, from about 5 to about 50 nucleotides, from about 5 to about 40 nucleotides, from about 5 to about 30 nucleotides, from about 5 to about 20 nucleotides, from about 5 to about 10 nucleotides, from about 10 to about 100 nucleotides, from about 10 to about 90 nucleotides, from about 10 to about 80 nucleotides, from about 10 to about 70 nucleotides, from about 10 to about 60 nucleotides, from about 10 to about 50 nucleotides, from about 10 to about 40 nucleotides, from about 10 to about 30 nucleotides, or from about 10 to about 20 nucleotides in length. In further embodiments, an oligonucleotide of the disclosure is, is about, is at least about, or is less than about 5, 10, 15, 20, 25, 30, 35, 40, 45, 50, 55, 60, 65, 70, 75, 80, 85, 90, 95, or 100 nucleotides in length.

### Ligation

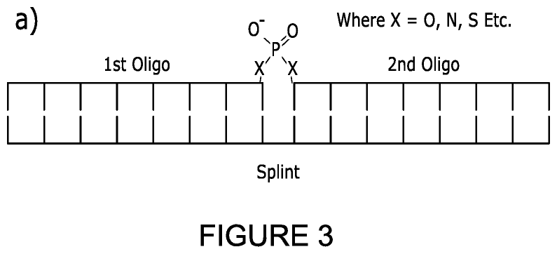

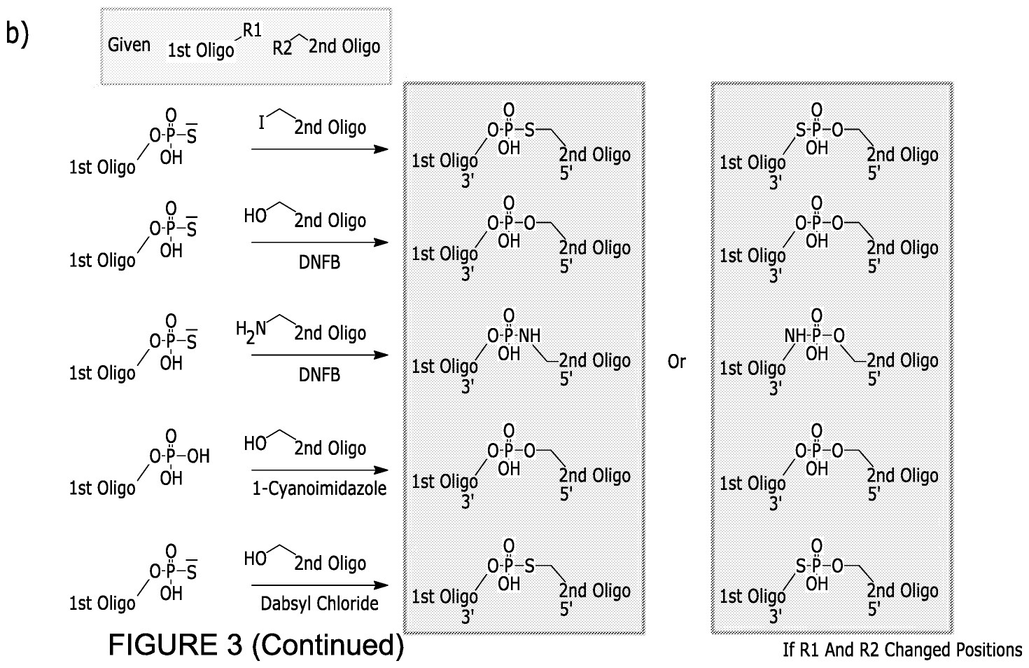

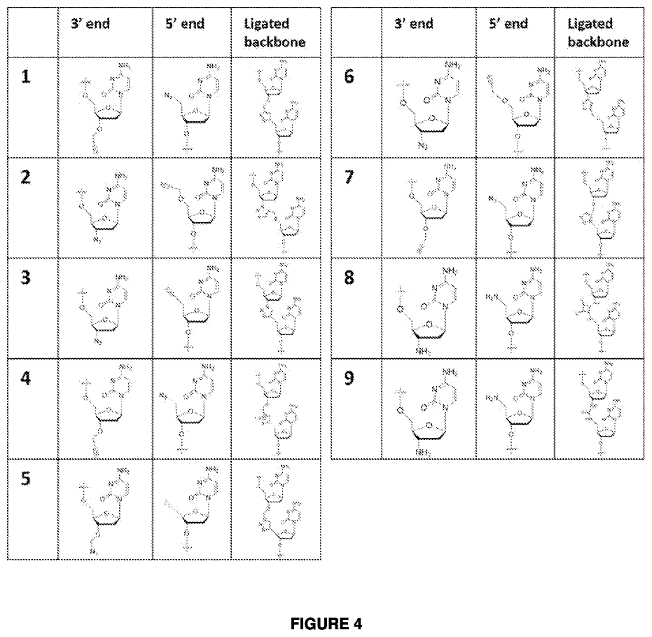

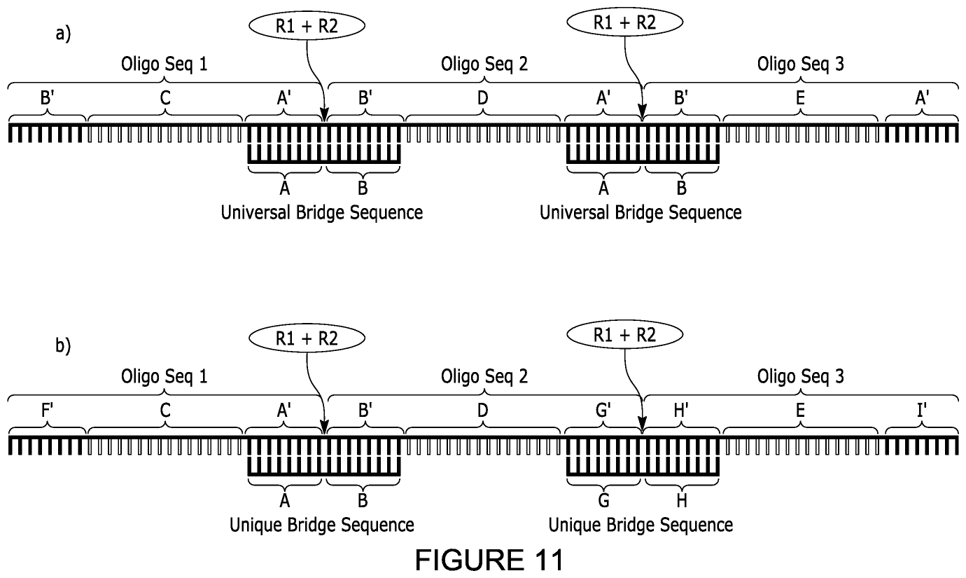

Any method for ligating oligonucleotides may be used in the methods of the disclosure. In any of the aspects or embodiments of the disclosure, chemical ligation is used to ligate oligonucleotides. In some embodiments, enzymatic ligation is used to ligate oligonucleotides. In any of the aspects or embodiments of the disclosure, a splint oligonucleotide is used during the ligation of oligonucleotides. A “splint oligonucleotide” is an oligonucleotide that improves the ligation efficiency of oligonucleotides. By way of example, when ligating two oligonucleotides together, the splint oligonucleotide hybridizes to an end of each oligonucleotide and the ends are subsequently ligated. See, for example, FIGS. 3a and 3b. In any of the aspects or embodiments of the disclosure, a splint oligonucleotide is used for each ligation in the method. In some embodiments, each splint oligonucleotide comprises a unique nucleotide sequence relative to every other splint oligonucleotide. See, e.g., FIG. 11b. In some embodiments, each splint oligonucleotide comprises a semi-random nucleotide sequence. In some embodiments, each oligonucleotide (e.g., barcode) used in a method of the disclosure comprises a nucleotide sequence such that a universal splint oligonucleotide may be used. As used herein, a “universal splint oligonucleotide” refers to a splint oligonucleotide in a population of splint oligonucleotides wherein each splint oligonucleotide in the population comprises or consists of the same nucleotide sequence. See, e.g., FIG. 11a.

In some embodiments, oligonucleotides (e.g., barcodes) comprise a modified backbone, such as a phosphorothiolate backbone. In various embodiments, reactions that may be performed to yield a phosphate derivative backbone are depicted in FIG. 3b, wherein the phosphate derivative group can either be on the 3′ end or 5′ end of either fragment.

In some embodiments, methods in which enzymatic ligation is used comprise use of a splint oligonucleotide. In some embodiments, methods in which chemical ligation is used comprise use of a splint oligonucleotide. In some embodiments, methods in which chemical ligation is used do not comprise use of a splint oligonucleotide. In some embodiments, variables that can increase ligation efficiency include, but are not limited to, temperature, buffer, divalent metals [Sci. Rep. 2014, 4, 4595], and the use of double-stranded DNA intercalators [(a) Nat. Commun. 2016, 6, 7304; (b) PNAS 2010, 107, 5288]. In various embodiments, prior to the chemical ligation a terminal deoxynucleotidyl transferase (TdT) enzyme is used to incorporate a 3′-modified nucleotide.

In some embodiments, chemically ligating oligonucleotides in the methods disclosed herein comprises synthesis of a 3′-5′ phosphodiester linkage. The phosphodiester linkage can be synthesized by methods known in the art. For example, the phosphodiester linkage can be synthesized by reacting a phosphate- or thiophosphate-terminated nucleoside with a hydroxyl-containing nucleoside. The reaction can be promoted by a suitable reagent, e.g., 1-fluoro-2,4-dinitrobenzene (DNFB) or 1-cyanoimidazole. In some embodiments, chemically ligating oligonucleotides in the methods disclosed herein comprises synthesis of an unnatural backbone linkage between two nucleosides. An “unnatural backbone linkage” is a linkage between two nucleotides other than a naturally-occurring 3′-5′ phosphodiester linkage. Non-limiting examples of unnatural backbone linkages include phosphoramidates, phosphorothiolates, triazoles, squaramides, and ureas. Unnatural backbone linkages can be synthesized by methods known in the art. For example, phosphoramidate linkages can be formed by reacting a terminal thiophosphate-modified nucleoside with an amine-modified nucleoside. The reaction can be promoted by a suitable reagent, e.g., 1-fluoro-2,4-dinitrobenzene (DNFB). Phosphorothiolate linkages can be formed by reacting a terminal thiophosphate-modified nucleoside with a hydroxyl-containing nucleoside. The reaction can be promoted by a suitable reagent, e.g., dabsyl chloride. Triazole linkages can be formed through an azide-alkyne cycloaddition reaction (e.g., a “click reaction”). Azide-alkyne cycloaddition reactions can be catalyzed (e.g., by Cu(I)) or uncatalyzed. Squaramide linkages can be formed by reacting amine-modified nucleosides with squaric acid or a nucleophilic derivative thereof. Squaramide formation can be promoted (e.g., using carbodiimide-mediated reaction conditions, such as using 1-ethyl-3-(3-dimethylaminopropyl) carbodiimide (EDC)), or unpromoted. Non-limiting examples of methods for synthesizing urea linkages include reacting an amine-modified nucleoside with an activating reagent (such as 1,1′-carbonyldiimidazole (CDI)), reacting an amine-modified nucleoside with an isocyanate-modified nucleoside, or reacting an amine-modified nucleoside with a carbamate-modified nucleoside.

### Illustrative Ligation Synthesis Workflows

As described herein, in some embodiments oligonucleotides comprise modified backbones. In order to utilize modified backbones, the methods require modified nucleotide bases at the 5′ and 3′ ends of each oligonucleotide sequence. There are multiple ways to include modified nucleotide bases to the ends of each oligonucleotide sequence.

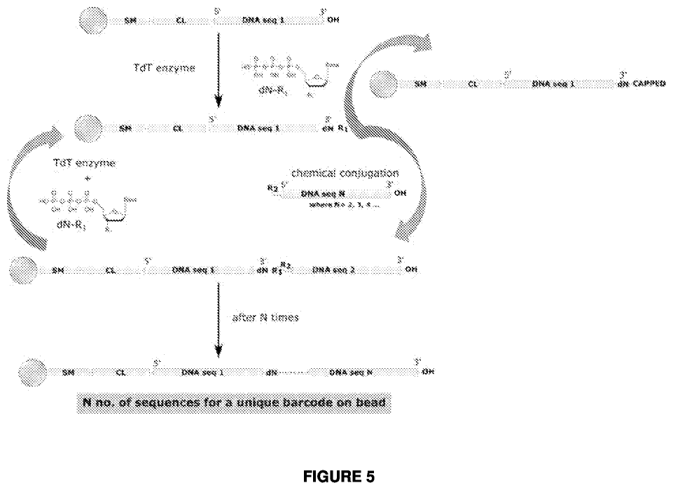

In some embodiments, modified nucleotide bases are added to the end of an oligonucleotide sequence using a terminal deoxynucleotidyl transferase (TdT) enzyme. In some embodiments, and as depicted in FIG. 5, the general steps of the ligation synthesis workflow comprise or consist of:
1. Conjugating a first oligonucleotide to solid phase particles (e.g., any particles that can be split from other particles and combined as a pool) that enable oligonucleotide synthesis, for example and without limitation controlled pore glass (CPG) or a bead;
2. Incorporating a 3′-modified nucleotide into the first oligonucleotide using TdT;
3. Chemically ligating a second oligonucleotide with 5′-orthogonal chemistry and the 3′-orthogonal chemistry of the first oligonucleotide;
4. Capping of any unreacted 3′-modified nucleotide that failed to ligate;
5. Repeating steps 2 to 4 until the nth round, where n=2, 3, 4, 5, 6, 7, 8, 9, 10, or more; and optionally
6. Cleaving the oligonucleotide (e.g., barcode) from the solid phase particle and optionally purifying it; and optionally
7. Conjugating the oligonucleotide (e.g., barcode) to a second solid phase particle or a solid support.

Before each new ligation in the foregoing workflow (i.e., prior to each time step (5) is performed), the pool is split. (See FIG. 1). One unique oligonucleotide will then be conjugated to each split-pool, and the diversity of the oligonucleotide sequence will increase, and will depend on the number of rounds of split-pool that are performed. In some embodiments, a washing step to wash off unbound material and/or excess reagents is performed after step (1), after step (2), after step (3), after step (4), after step (5), and/or after step (7).

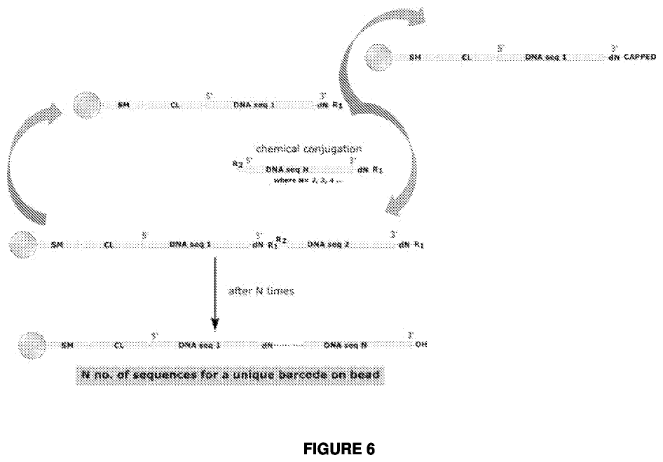

In some embodiments, modified nucleotide bases are added to the end of an oligonucleotide sequence in the absence of a terminal deoxynucleotidyl transferase (TdT) enzyme. In some embodiments, and as depicted in FIG. 6, the general steps of the ligation synthesis workflow comprise or consist of:
1. Conjugating a first oligonucleotide to a solid phase particles (e.g., any particles that can be split from other particles and combined as a pool) that enable oligonucleotide synthesis, for example and without limitation controlled pore glass (CPG) or a bead;
2. Chemically ligating a second oligonucleotide with 5′-orthogonal chemistry and the 3′-orthogonal chemistry of the first oligonucleotide;
3. Capping of any unreacted 3′-modified nucleotide that failed to ligate;
4. Repeating steps 2 to 3 until the nth round, where n=2, 3, 4, 5, 6, 7, 8, 9, 10, or more; and optionally
5. Cleaving the oligonucleotide (e.g., barcode) from the solid phase particles and optionally purifying it; and optionally
6. Conjugating the oligonucleotide (e.g., barcode) to a second solid phase particle or a solid support.

Before each new ligation in the foregoing workflow (i.e., prior to each time step (3) is performed), the pool is split. (See FIG. 1). One unique oligonucleotide will then be conjugated to each split-pool, and the diversity of the oligonucleotide sequence will increase, and will depend on the number of rounds of split-pool that are performed. In some embodiments, a washing step to wash off unbound material and/or excess reagents is performed after step (1), after step (2), after step (3), after step (4), and/or after step (6).

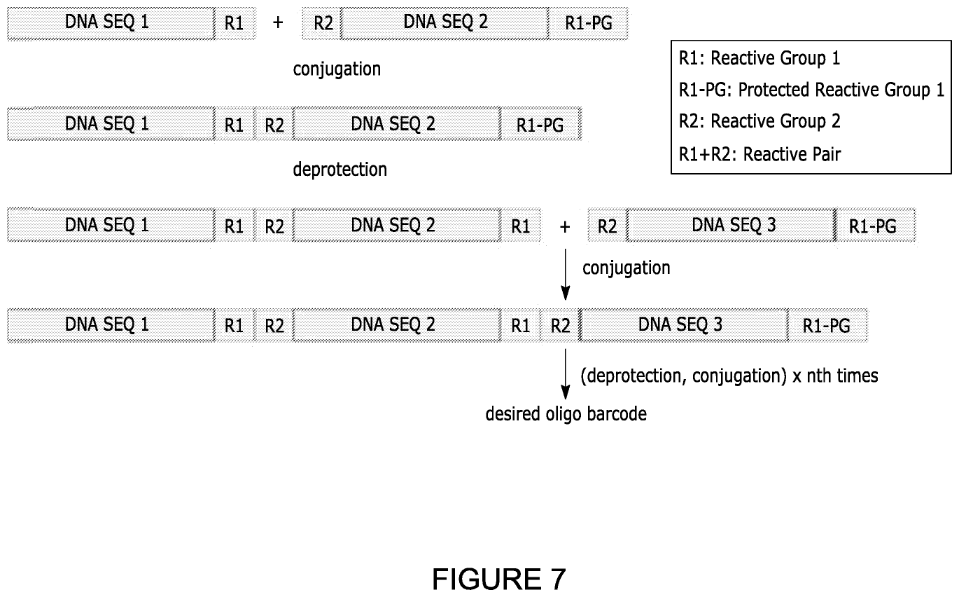

For any of the ligation workflows described herein, there are optional additional steps that may be performed to improve conjugation efficiency and yield. In some embodiments, if R1 and R2 (as depicted in, e.g., FIGS. 5 and 6) are the only two reactive groups used in the workflow, R1 on the second oligonucleotide sequence can be capped with a protecting group while R2 of the second oligonucleotide sequence is conjugated to R1 of the first oligonucleotide sequence. Upon completion of the conjugation, R1 of the second oligonucleotide sequence is then deprotected to enable conjugation of the third oligonucleotide sequence, and the cycle continues. (See FIG. 7).

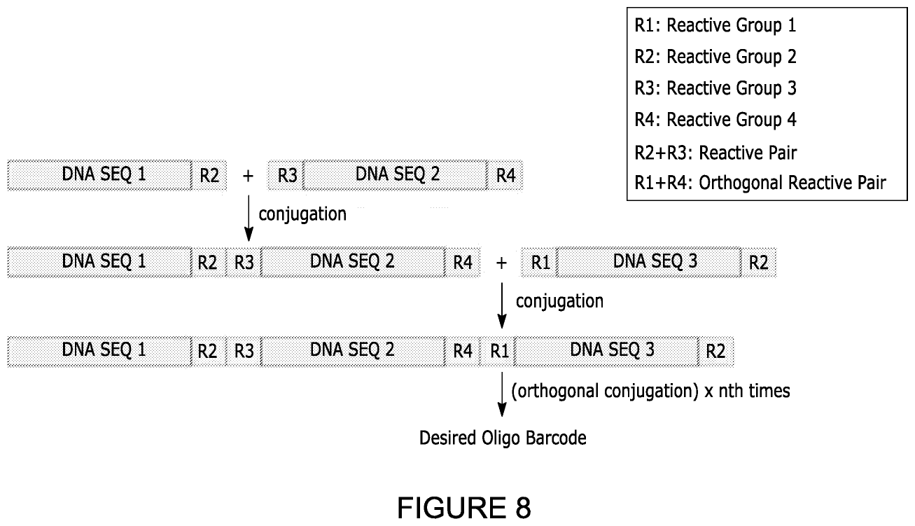

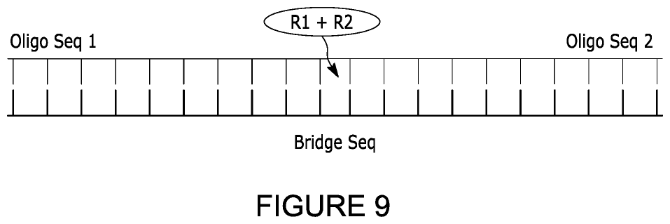

Alternatively, in some embodiments the number of reactive groups is expanded to four (i.e., R1, R2, R3, R4). By way of example, the first oligonucleotide may have R1 and R2 reactive handles with R1 either being capped or conjugated on a solid support. The second oligonucleotide may have R3 and R4 reactive handles. R2 of the first oligonucleotide can then conjugate to R3 of the second oligonucleotide. Subsequently, the R4 of second oligonucleotide can then conjugate to R1 of the third oligonucleotide that has similar reactive entities as the first oligonucleotide. Here, R2+R3 is orthogonal to R1+R4. (See FIG. 8).

To minimize undesired barcodes, in some embodiments a capping step is included to terminate the synthesis of failed ligation for any of the workflows of the disclosure. While the workflows described herein focus on solid-support chemical ligation, it is further contemplated that the ligation synthesis may also be performed in solution prior to bead attachment. Additionally, while the workflows described herein focus on synthesis prior to bead attachment, in some embodiments the ligation synthesis may also be performed directly on the bead.

To further illustrate the present disclosure, examples are given herein. It is to be understood that these examples are provided for illustrative purposes and are not to be construed as limiting the scope of the present disclosure.

### EXAMPLES

### Example 1

Synthesis of exemplary modified nucleotides for ligation.

Synthesis of 3′-alkyne dT triphosphate:

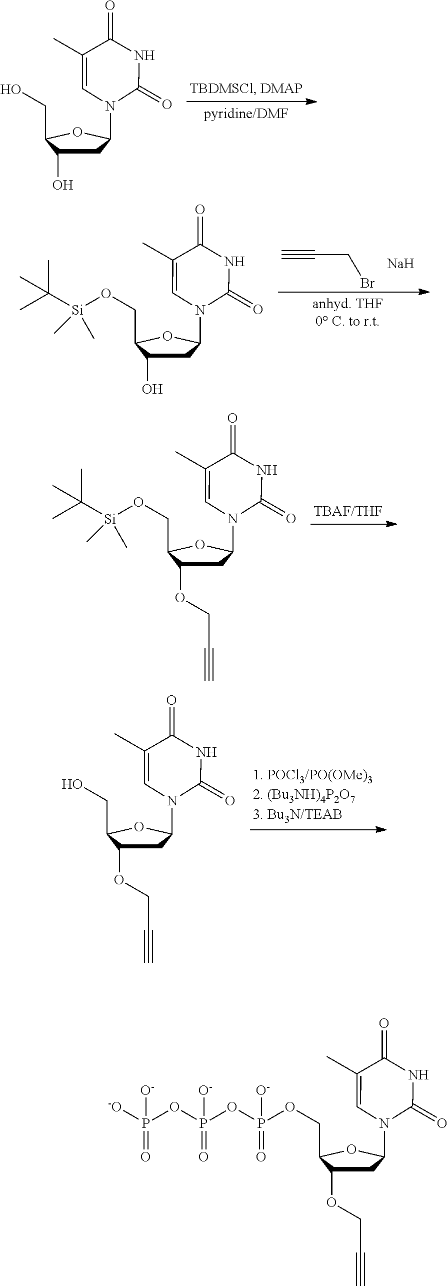

Synthesis of 3′-alkyne dA triphosphate:

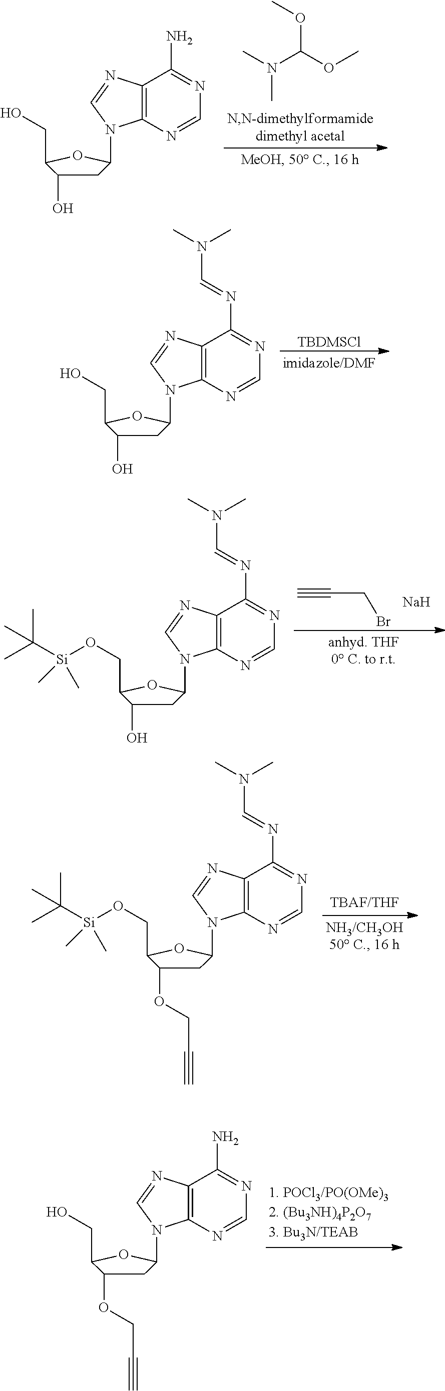

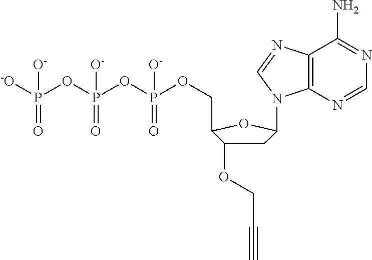

In this example, two 3′-alkyne modified deoxyribonucleotides useful in the chemical ligation methods disclosed herein were synthesized. These modified deoxyribonucleotides can be used to produce a triazole linkage (i.e., an unnatural backbone linkage) between nucleotides in a chemical ligation as disclosed herein.

Synthesis of 3′-alkyne dT triphosphate: briefly, deoxythymidine was treated with tert-butyldimethylsilyl chloride (TBDMSCI) in pyridine/DMF to protect the 5′ hydroxyl group. The silylated deoxythymidine was then treated with propargyl bromide and sodium hydride in anhydrous THF in order to yield 3′-alkyne deoxythymidine. Desilylation with tetrabutylammonium fluoride (TBAF) in THF, followed by stepwise treatment with phosphoryl chloride/trimethyl phosphite (POCl₃/PO(OMe)₃), tributylammonium pyrophosphate ((Bu₃NH)₄P₂O₇), and Bu₃N/TEAB yielded the desired 3′-alkyne dT triphosphate.

Synthesis of 3′-alkyne dA triphosphate: briefly, deoxyadenosine was treated with N, N-dimethylformamide dimethyl acetal to yield 10-N,N-dimethylformamidine-protected deoxyadenosine. The protected dA was treated with tert-butyldimethylsilyl chloride (TBDMSCI) in pyridine/DMF to protect the 5′ hydroxyl group. The silylated deoxyadenosine was then treated with propargyl bromide and sodium hydride in anhydrous THF in order to yield 3′-alkyne deoxyadenosine. Desilylation with tetrabutylammonium fluoride (TBAF) in THF, followed by stepwise treatment with phosphoryl chloride/trimethyl phosphite (POCl₃/PO(OMe) 3), tributylammonium pyrophosphate ((Bu₃NH)₄P₂O₇), and Bu₃N/TEAB yielded the desired 3′-alkyne dA triphosphate.

### Example 2

In this example, an array of the disclosure is generated. Array generation begins by synthesizing a first set of 100 unique oligonucleotide beads. Next, another 100 unique oligonucleotide barcodes is chemically ligated on at each split-pool step. After three rounds of split-pool and ligation, 100,000,000 barcoded bead pool is generated. Chemical ligation can occur on beads or in solution. If in solution, the resulting oligonucleotide is chemically attached to beads after completing all ligation cycles.

Three unique oligonucleotide sequences with a 5′-terminal amine or hydrazine, and a cleavable linker (FIG. 10), are first synthesized on an oligosynthesizer and purified. Each of these unique oligonucleotide sequences are then chemically attached through the 5′ end to 3 pools of aldehyde-functionalized beads, i.e., 1 unique oligonucleotide sequence to one of the three pools of beads. Sodium cyanoborohydride is then added to reduce the imine bond formed between the 5′-terminal hydrazine and aldehyde-functionalized beads. After oligonucleotide attachment, excess aldehyde on the beads are capped using any small molecule amine, and further reduced with sodium cyanoborohydride. The beads from three pools are then combined. TdT enzyme and 3′-alkyne modified nucleotide is then added to the combined bead pools. Upon the addition of one 3′-alkyne modified nucleotide to the 3′OH end of oligonucleotide attached to beads, TdT enzyme and the 3′-alkyne modified nucleotide are washed away using buffer. This combined pool is then now randomly split into three new pools. To each of the three new pools of beads, a unique oligonucleotide sequence with 5′-azide is added to each pool. Click ligation is performed with or without splint. Upon completion of ligation, the beads are washed multiple times with urea in buffer and buffer to dehybridize any splint and remove unreacted oligonucleotides. Unreacted 3′-alkyne is capped using a small molecule azide and washed. Another round of TdT enzyme and 3′-alkyne modified nucleotide is then added to enable the ligation of the third oligonucleotide sequence to the 3′OH end of the second oligonucleotide sequence. The process of TdT enzyme and 3′-alkyne modified nucleotide addition, wash and oligonucleotide ligation, with capping steps, is repeated until the desired oligonucleotide barcode length is obtained.

### Example 3

In this example, the methods of the disclosure are used for spatial applications. The barcodes are generated on beads and either (1) affixed (e.g., via hybridization to a surface primer) directly to a solid support (such as a flow cell), (2) the oligonucleotides are released from beads and attached to new beads after purification; the beads then are affixed (e.g., via hybridization to a surface primer) to a solid support, or (3) the oligonucleotides are released from beads and affixed (e.g., via hybridization to a surface primer) to a solid support. The tissue is then placed on the solid support and nucleic acid from the tissue is captured by the oligonucleotides (e.g., mRNA capture by the poly(T) capture sequence). The captured nucleic acid can be sequenced in situ or ex situ. For mRNA, cDNA is generated on the solid support.

What is claimed is:

1. A method of generating a bead array comprising:
(i) attaching a first oligonucleotide to a first population of beads, attaching a second oligonucleotide to a second population of beads, and attaching a third oligonucleotide to a third population of beads, wherein the first oligonucleotide, the second oligonucleotide, and the third oligonucleotide comprise unique nucleotide sequences relative to each other;
(ii) combining the first population of beads, the second population of beads, and the third population of beads to create a first pool;
(iii) splitting the first pool into a first plurality of aliquots;
(iv) in a first aliquot of the first plurality of aliquots, chemically ligating a fourth oligonucleotide to:
the first oligonucleotide in the presence of a first splint oligonucleotide, wherein the first splint oligonucleotide hybridizes to both the fourth oligonucleotide and to the first oligonucleotide;
the second oligonucleotide in the presence of a second splint oligonucleotide, wherein the second splint oligonucleotide hybridizes to both the fourth oligonucleotide and to the second oligonucleotide; and
the third oligonucleotide in the presence of a third splint oligonucleotide, wherein the third splint oligonucleotide hybridizes to both the fourth oligonucleotide and to the third oligonucleotide;
wherein the first splint oligonucleotide, the second splint oligonucleotide, and the third splint oligonucleotide comprise unique nucleotide sequences relative to each other;
in a second aliquot of the first plurality of aliquots, chemically ligating a fifth oligonucleotide to:
the first oligonucleotide in the presence of a fourth splint oligonucleotide, wherein the fourth splint oligonucleotide hybridizes to both the fifth oligonucleotide and to the first oligonucleotide;
the second oligonucleotide in the presence of a fifth splint oligonucleotide, wherein the fifth splint oligonucleotide hybridizes to both the fifth oligonucleotide and to the second oligonucleotide; and
the third oligonucleotide in the presence of a sixth splint oligonucleotide, wherein the sixth splint oligonucleotide hybridizes to both the fifth oligonucleotide and to the third oligonucleotide;
wherein the fourth splint oligonucleotide, the fifth splint oligonucleotide, and the sixth splint oligonucleotide comprise unique nucleotide sequences relative to each other; and
in a third aliquot of the first plurality of aliquots, chemically ligating a sixth oligonucleotide to:
the first oligonucleotide in the presence of a seventh splint oligonucleotide, wherein the seventh splint oligonucleotide hybridizes to both the sixth oligonucleotide and to the first oligonucleotide;
the second oligonucleotide in the presence of an eighth splint oligonucleotide, wherein the eighth splint oligonucleotide hybridizes to both the sixth oligonucleotide and to the second oligonucleotide; and
the third oligonucleotide in the presence of a ninth splint oligonucleotide, wherein the ninth splint oligonucleotide hybridizes to both the sixth oligonucleotide and to the third oligonucleotide,
wherein the seventh splint oligonucleotide, the eighth splint oligonucleotide, and the ninth splint oligonucleotide comprise unique nucleotide sequences relative to each other;
(v) combining the first aliquot of the first plurality of aliquots, the second aliquot of the first plurality of aliquots, and the third aliquot of the first plurality of aliquots to create a second pool;
(vi) splitting the second pool into a second plurality of aliquots;
(vii) in a first aliquot of the second plurality of aliquots, chemically ligating a seventh oligonucleotide to the fourth oligonucleotide in the presence of a tenth splint oligonucleotide, wherein the tenth splint oligonucleotide hybridizes to both the seventh oligonucleotide and to the fourth oligonucleotide;
in a second aliquot of the second plurality of aliquots, chemically ligating an eighth oligonucleotide to the fifth oligonucleotide in the presence of an eleventh splint oligonucleotide, wherein the eleventh splint oligonucleotide hybridizes to both the eighth oligonucleotide and to the fifth oligonucleotide; and
in a third aliquot of the second plurality of aliquots, chemically ligating a ninth oligonucleotide to the sixth oligonucleotide in the presence of a twelfth splint oligonucleotide, wherein the twelfth splint oligonucleotide hybridizes to both the ninth oligonucleotide and to the sixth oligonucleotide, and wherein the tenth splint oligonucleotide, the eleventh split oligonucleotide, and the twelfth splint oligonucleotide comprise unique nucleotide sequences relative to each other.

2. A method of generating a bead array comprising:
(i) attaching a first oligonucleotide to one or more beads in a first population of beads, attaching a second oligonucleotide to one or more beads in a second population of beads, and attaching a third oligonucleotide to one or more beads in a third population of beads, wherein the first oligonucleotide, the second oligonucleotide, and the third oligonucleotide comprise unique nucleotide sequences relative to each other;
(ii) combining the first population of beads, the second population of beads, and the third population of beads to create a first pool;
(iii) splitting the first pool into a first plurality of aliquots;
(iv) in a first aliquot of the first plurality of aliquots, chemically ligating a fourth oligonucleotide to the first oligonucleotide, the second oligonucleotide, and the third oligonucleotide; in a second aliquot of the first plurality of aliquots, chemically ligating a fifth oligonucleotide to the first oligonucleotide, the second oligonucleotide, and the third oligonucleotide; and in a third aliquot of the first plurality of aliquots, chemically ligating a sixth oligonucleotide to the first oligonucleotide, the second oligonucleotide, and the third oligonucleotide;
(v) combining the first aliquot of the first plurality of aliquots, the second aliquot of the first plurality of aliquots, and the third aliquot of the first plurality of aliquots to create a second pool;
(vi) splitting the second pool into a second plurality of aliquots;
(vii) in a first aliquot of the second plurality of aliquots, chemically ligating a seventh oligonucleotide to the fourth oligonucleotide; in a second aliquot of the second plurality of aliquots, chemically ligating an eighth oligonucleotide to the fifth oligonucleotide; and in a third aliquot of the second plurality of aliquots, chemically ligating a ninth oligonucleotide to the sixth oligonucleotide.

3. The method of claim 1, further comprising:
(viii) combining the first aliquot of the second plurality of aliquots, the second aliquot of the second plurality of aliquots, and the third aliquot of the second plurality of aliquots to create a third pool;
(ix) splitting the third pool into a third plurality of aliquots;
(x) in a first aliquot of the third plurality of aliquots, chemically ligating a tenth oligonucleotide to the seventh oligonucleotide in the presence of a thirteenth splint oligonucleotide, wherein the thirteenth splint oligonucleotide hybridizes to both the tenth oligonucleotide and to the seventh oligonucleotide;
in a second aliquot of the third plurality of aliquots, chemically ligating an eleventh oligonucleotide to the eighth oligonucleotide in the presence of a fourteenth splint oligonucleotide, wherein the fourteenth splint oligonucleotide hybridizes to both the eleventh oligonucleotide and to the eighth oligonucleotide; and
in a third aliquot of the third plurality of aliquots, chemically ligating a twelfth oligonucleotide to the ninth oligonucleotide in the presence of a fifteenth splint oligonucleotide, wherein the fifteenth splint oligonucleotide hybridizes to both the twelfth oligonucleotide and to the ninth oligonucleotide, and wherein the thirteenth splint oligonucleotide, the fourteenth splint oligonucleotide, and the fifteenth splint oligonucleotide comprise unique nucleotide sequences relative to each other.

4. The method of claim 2, further comprising:
(viii) combining the first aliquot of the second plurality of aliquots, the second aliquot of the second plurality of aliquots, and the third aliquot of the second plurality of aliquots to create a third pool;
(ix) splitting the third pool into a third plurality of aliquots;
(x) in a first aliquot of the third plurality of aliquots, chemically ligating a tenth oligonucleotide to the seventh oligonucleotide; in a second aliquot of the second plurality of aliquots, chemically ligating an eleventh oligonucleotide to the eighth oligonucleotide; and in a third aliquot of the second plurality of aliquots, chemically ligating a twelfth oligonucleotide to the ninth oligonucleotide.

5. The method of any one of claims 1-4, wherein the method results in the bead array comprising at least about 1×10⁶ unique oligonucleotide sequences attached thereto.

6. The method of claim 5, wherein the method results in the bead array comprising at least about 1×10⁷ unique oligonucleotide sequences attached thereto.

7. The method of claim 5 or claim 6, wherein the method results in the bead array comprising at least about 1×10⁸ unique oligonucleotide sequences attached thereto.

8. The method of claim 3, wherein the first, second, third, fourth, fifth, sixth, seventh, eighth, ninth, tenth, eleventh, twelfth, thirteenth, fourteenth, and fifteenth splint oligonucleotides have the same nucleotide sequence.

9. The method of any one of claims 1-8, wherein an oligonucleotide comprising (i) a poly(T) or poly(A) nucleotide sequence; (ii) one or more gene-specific capture sequences; (iii) one or more universal capture sequences (e.g., a random nucleotide sequence or a semi-random nucleotide sequence), or a combination thereof is chemically ligated to the end of each oligonucleotide on each bead in the bead array.

10. The method of any one of claims 1-9, wherein (i) comprises about 100 populations of beads, each population of beads comprising unique oligonucleotides attached thereto relative to every other population of beads.

11. The method of any one of claims 1-10, wherein (i) comprises about 13,600 populations of beads, and each population of beads comprises unique oligonucleotides attached thereto relative to every other population of beads.

12. The method of any one of claims 1-11, wherein the first oligonucleotide, the second oligonucleotide, and/or the third oligonucleotide are attached to the one or more beads through a cleavable linker.

13. The method of any one of claims 1-12, wherein prior to the chemically ligating a terminal deoxynucleotidyl transferase (TdT) enzyme is used to incorporate a 3′-modified nucleotide.

14. The method of any one of claims 1-12, wherein one or more of the first oligonucleotide, the second oligonucleotide, the third oligonucleotide, the fourth oligonucleotide, the fifth oligonucleotide, the sixth oligonucleotide, the seventh oligonucleotide, the eighth oligonucleotide, or the ninth oligonucleotide is a barcode.

15. The method of any one of claims 1-14, wherein each of the first oligonucleotide, the second oligonucleotide, the third oligonucleotide, the fourth oligonucleotide, the fifth oligonucleotide, the sixth oligonucleotide, the seventh oligonucleotide, the eighth oligonucleotide, and the ninth oligonucleotide is a barcode.

16. The method of any one of claims 3-15, wherein one or more of the tenth oligonucleotide, the eleventh oligonucleotide, or the twelfth oligonucleotide is a barcode.

17. The method of any one of claims 3-15, wherein each of the tenth oligonucleotide, the eleventh oligonucleotide, and the twelfth oligonucleotide is a barcode.

18. The method of any one of claims 14-17, wherein the barcode is a spatial barcode.
